(19) United States
(12) Patent Application Publication
(10) Pub. No.: US 2007/0105125 A1
(43) Pub. Date: May 10, 2007

(54) Chemical array housing having a gas delivery element and methods of using the same

(76) Inventors: George P. Tsai, San Jose, CA (US); Jeffrey M. McMillan, Morgan Hill, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/270,764

(22) Filed: Nov. 8, 2005

(51) Int. Cl. C12Q 1/68; C12M 3/00

(52) U.S. Cl. 435/6; 435/287.2

(57) ABSTRACT

Chemical array housings configured for use with chemical array readers are provided, where the housings include at least one chemical array holding element and a gas delivery element. Aspects of the invention include positioning a chemical array in the housing and introducing a gas into the housing. Also provided are chemical array readers that include housings of the present invention. The subject devices and methods find use in a variety of different applications, including chemical array reading applications.

### INTRODUCTION

Optical readers or scanners play an important role in many array based applications. For instance, optical readers act like a large field fluorescence microscope by employing laser induced fluorescence to determine the fluorescent pattern caused by the binding of labeled molecules on the surface of an array that is scanned. The pattern of binding by target molecules to biopolymer probe features or spots on the substrate produces a pattern on the surface of the substrate and provides desired information about the sample. In most instances, the target molecules are labeled with a detectable tag such as a fluorescent tag or chemiluminescent tag. The resultant binding interaction or complexes of binding pairs are then detected and read or interrogated, for example by an optical reader.

In such devices, a laser light source generates a collimated beam that is focused on the array and sequentially illuminates small surface regions of known location on the array substrate to excite fluorescent tags, generating a signal only in those spots on the biochip that have a target molecule and thus a fluorescent tag bound to a probe molecule. The resulting fluorescence signals from the surface regions are collected and transmitted to one or more optical detectors. A recording device, such as a computer memory, records the detected signals and builds up a scan file of intensities as a function of position, or time as it relates to the position. In this way, a laser induced fluorescence reader provides for analyzing large numbers of different target molecules (e.g., ligands) of interest, e.g., genes/mutations/alleles, in a biological sample located on one or more arrays.

In many array-based assays, fluorescent labels are employed to label target molecules that are bound to surface immobilized probes of the array. Representative fluorescent labels that find use in various array protocols currently practiced in the art include xanthene dyes, e.g. fluorescein and rhodamine dyes, such as fluorescein isothiocyanate (FITC), 6-carboxyfluorescein (commonly known by the abbreviations FAM and F),6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy- X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g. Cy3, Cy5 and Cy7 dyes; coumarins, e.g. umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes; quinoline dyes; and benzopyrylium-based fluorescent dyes.

While fluorescent labels are used frequently in array-based applications, it is well recognized in the art that the fluorescent dyes are susceptible to degradation and loss of fluorescent activity due to their relatively elevated chemical reactivity. Such degradation can have a significant adverse impact on the results obtained from a given assay. For example, in a two color assay where two distinguishably fluorescent labeled samples are compared, one of the fluorescent dyes may be degraded to an extent greater than the other, leading to significant inaccuracies in the observed results of the assay.

Label degradation has been attributed, at least in part, to the presence of ozone. As such, numerous different approaches have been developed in order to limit the amount of ozone that comes in contact with the label. However, disadvantages exist with each of the currently employed approaches.

There is, therefore, a continued need for the identification of new ways to limit exposure of labels present on an array surface to ozone.

### SUMMARY OF THE INVENTION

Chemical array housings configured for use with chemical array readers are provided, where the housings include at least one chemical array holding element and a gas delivery element. Aspects of the invention include positioning a chemical array in the housing and introducing a gas into the housing. Also provided are chemical array readers that include housings of the present invention. The subject devices and methods find use in a variety of different applications, including chemical array reading applications.

### BRIEF DESCRIPTION OF THE FIGURES

FIG. 1 illustrates a substrate carrying multiple arrays.

FIG. 2 is an enlarged view of a portion of FIG. 1 showing multiple ideal spots or features.

FIG. 3 is an enlarged illustration of a portion of the substrate in FIG. 2.

FIG. 4A is a perspective view of a chemical array housing that includes a chemical array container element and a lid of the subject invention, wherein the lid is removed from the container element.

FIG. 4B is a top-down view of a chemical array housing that includes a chemical array container element and a lid, wherein the lid is fitted to the top of the container element.

FIG. 4C is a bottom view of a lid in accordance with the subject invention.

FIG. 5A is a perspective view of a chemical array slide that is inserted into a slide holder for use in conjunction with the subject invention.

FIG. 5B is a perspective view of the chemical array slide and slide holder of FIG. 5A as it is inserted into a chemical array containing element in accordance with the subject invention.

FIG. 6 is schematic representation depicting an apparatus in accordance with the present invention.

### DEFINITIONS

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

“Ligands” are moieties that specifically bind to analytes of interest, where in representative embodiments ligands are polymers.

The term “peptide” as used herein refers to any polymer compound produced by amide formation between an α-carboxyl group of one amino acid and an α-amino group of another group.

The term “oligopeptide” as used herein refers to peptides with fewer than about 10 to 20 residues, i.e. amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single-stranded nucleotide multimers of from about 10 to about 100 nucleotides and up to 200 nucleotides in length.

The term “polynucleotide” as used herein refers to single- or double-stranded polymers composed of nucleotide monomers of generally greater than about 100 nucleotides in length.

The term “Oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In the practice of the instant invention, oligomers will generally comprise about 2-50 monomers, preferably about 2-20, more preferably about 3-10 10 monomers.

The term “monomer” as used herein refers to a chemical entity that can be covalently linked to one or more other such entities to form a polymer. Of particular interest to the present application are nucleotide “monomers” that have first and second sites (e.g., 5′0 and 3′sites) suitable for binding to other like monomers by means of standard chemical reactions (e.g., nucleophilic substitution), and a diverse element which distinguishes a particular monomer from a different monomer of the same type (e.g., a nucleotide base, etc.). In the art synthesis of nucleic acids of this type utilizes an initial substrate-bound monomer that is generally used as a building-block in a multi-step synthesis procedure to form a complete nucleic acid.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, -alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “amino acid” is intended to include not only the L, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, ε-nicotinoyl-lysine, isonipecotic acid, tetrahydroisoquinoleic acid, α-aminoisobutyric acid, sarcosine, citrulline, cysteic acid, t-butylglycine, t-butylalanine, phenylglycine, cyclohexylalanine, β-alanine, 4-aminobutyric acid, and the like.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

The term “sample” as used herein relates to a material or mixture of materials, typically, although not necessarily, in fluid form, containing one or more components of interest.

An “array,” or “chemical array’used interchangeably includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of ligand polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′or 5′terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges.

At least some, or all, of the features are of different compositions (for example, when any repeats of each feature composition are excluded, the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm^{2,} 10 cm² or 1 cm². In representative embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light.

Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays may be fabricated using drop deposition from pulse jets of either precursor units (such as nucleotide or amino acid monomers) in the case of in situ fabrication, or the previously obtained biomolecule, e.g., polynucleotide. Such methods are described in detail in, for example, the previously cited references including U.S. Pat No. 6,242,266, U.S. Pat No. 6,232,072, U.S. Pat No. 6,180,351, U.S. Pat No. 6,171,797, U.S. Pat No. 6,323,043, U.S. patent application Ser. No. 09/302,898 filed Apr. 30, 1999 by Caren et al., and the references cited therein. Other drop deposition methods can be used for fabrication, as previously described herein.

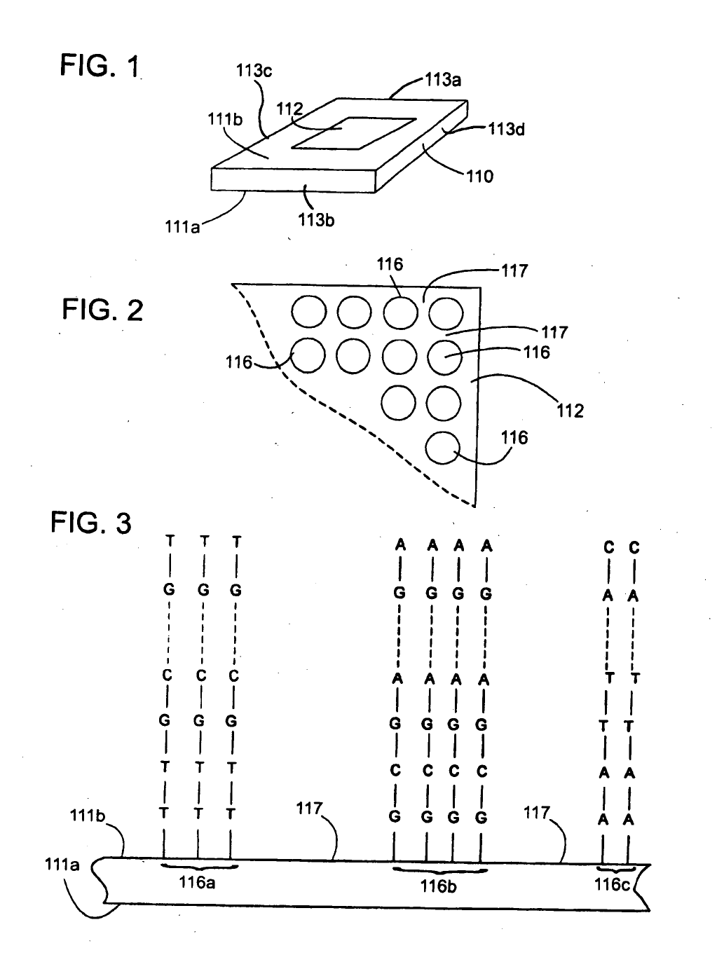

An exemplary chemical array is shown in FIGS. 1-3, where the array shown in this representative embodiment includes a contiguous planar substrate 110 carrying an array 112 disposed on a surface 111b of substrate 110. It will be appreciated though, that more than one array (any of which are the same or different) may be present on surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the surface 111b, with regions of the rear surface 111b adjacent the opposed sides 113c, 113d and leading end 113a and trailing end 113b of slide 110, not being covered by any array 112. A second surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymer ligands, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In those embodiments where an array includes two or more features immobilized on the same surface of a solid support, the array may be referred to as addressable. An array is “addressable” when it has multiple regions of different moieties (e.g., different polynucleotide sequences) such that a region (e.g., a “feature” or “spot” of the array) at a particular predetermined location (e.g., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An array “assembly” includes a substrate and at least one chemical array, e.g., on a surface thereof. Array assemblies may include one or more chemical arrays present on a surface of a device that includes a pedestal supporting a plurality of prongs, e.g., one or more chemical arrays present on a surface of one or more prongs of such a device. “Array unit” may be used interchangeably with “array assembly”.

The term “substrate” as used herein refers to a surface upon which ligands molecules or probes, e.g., an array, may be adhered. Glass slides are the most common substrate for biochips, although fused silica, silicon, plastic and other materials are also suitable. In representative embodiments, the substrate includes silica.

When two or more items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known. A “web” references a long continuous piece of substrate material having a length greater than a width. For example, the web length to width ratio may be at least 5/1, 10/1, 50/1, 100/1, 200/1, or 500/1, or even at least 1000/1.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

“Rigid” refers to a material or structure which is not flexible, and is constructed such that a segment about 2.5 by 7.5 cm retains its shape and cannot be bent along any direction more than 60 degrees (and often not more than 40, 20, 10, or 5 degrees) without breaking.

“Hybridizing” and “binding”, with respect to polynucleotides, are used interchangeably. The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO_{4,} 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser.No. 09/655,482 filed on Sep.5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

“Contacting” means to bring or put together. As such, a first item is contacted with a second item when the two items are brought or put together, e.g., by touching them to each other.

“Depositing” means to position or place an item at a location-or otherwise cause an item to be so positioned or placed at a location. Depositing includes contacting one item with another. Depositing may be manual or automatic, e.g., “depositing” an item at a location may be accomplished by automated robotic devices.

By “remote location,” it is meant a location other than the location at which the array (or referenced item) is present and hybridization occurs (in the case of hybridization reactions). For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information means transmitting the data representing that information as signals (e.g., electrical, optical, radio signals, and the like) over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination which will perform the functions required of it. For example, any processor herein may be a programmable digital microprocessor such as available in the form of a electron controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

A “reader” or “scanner” is a device for evaluating arrays. In readers, an optical light source, particularly a laser light source, generates a collimated beam. The collimated beam is focused on the array and sequentially illuminates small surface regions of known location (i.e. a position) on an array substrate. The resulting signals from the surface regions are collected either confocally (employing the same lens used to focus the light onto the array) or off-axis (using a separate lens positioned to one side of the lens used to focus the beam onto the array). The collected signals are then transmitted through appropriate spectral filters, to an optical detector. A recording device, such as a computer memory, records the detected signals and builds up a raster scan file of intensities as a function of position, or time as it relates to the position. Such intensities, as a function of position, are typically referred to in the art as “pixels”. Biopolymer arrays are often scanned and/or scan results are often represented at 5 or 10 micron pixel resolution. To achieve the precision required for such activity, components such as the lasers must be set and maintained with particular alignment. Scanners may be bidirectional, or unidirection, as is known in the art.

In representative embodiments, the reader used for the evaluation of arrays includes a scanning fluorometer. A number of different types of such devices are commercially available from different sources, such as Perkin-Elmer, Agilent, Packard, Axon Instruments, etc., and examples of typical scanners are described in U.S. Pat. Nos. 5,091,652; 5,760,951, 6,320,196 and 6,355,934.

An array “package” may be the array plus only a substrate on which the array is deposited, although the package may include other features (such as a housing with a chamber).

A “chamber” references an enclosed volume (although a chamber may be accessible through one or more ports). It will also be appreciated that throughout the present application, that words such as “top,” “upper,” and “lower” are used in a relative sense only.

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

### DETAILED DESCRIPTION OF REPRESENTATIVE EMBODIMENTS

Chemical array housings configured for use with chemical array readers are provided, where the housings include at least one chemical array holding element and a gas delivery element. Aspects of the invention include positioning a chemical array in the housing and introducing a gas into the housing. Also provided are chemical array readers that include housings of the present invention. The subject devices and methods find use in a variety of different applications, including chemical array reading applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

An aspect of the present invention is a chemical array housing that is configured so that a gas can be delivered into the housing. By “chemical array housing” is meant a structure that includes at least a container element. The container element has at least one chemical array holding element. In addition, the housing includes a lid element that fits onto the container element to provide an at least partially enclosed housing. A feature of the housing is the presence of a gas delivery element, which may be associated with either the container or lid elements of the housing. The interior of the housing may or may not be sealed in a vapor and fluid tight manner from the environment exterior of the housing.

A container element of the subject invention may be of any shape and size so long as it is configured to contain one or more chemical array holding elements that are adapted for receiving and holding a chemical array. For instance, a chemical array container element may be circular, rectangular, square, or the like. In one embodiment, as will be described in greater detail below, a chemical array container element of the subject invention is circular, for instance, in the shape of a chemical array carousel.

A feature of the chemical array container element is that it is adapted for housing one or more chemical array holding elements. Accordingly, the size and shape of the chemical array container element will vary depending upon the size and shape of the chemical array holding elements included therein, as will be described in better detail below. For instance, in certain embodiments the chemical array container element may be a rectangular unit or a circular unit with a length or diameter that is proportional to the number and dimension of the included chemical array holding elements, the dimensions of which are in turn dependent upon the number and size of the chemical arrays or chemical array slide holder elements to be contained therein.

By “chemical array holding element” is meant an element within a chemical array container element that is configured for receiving and holding a chemical array and/or chemical array slide holder. In one embodiment, the chemical array holding element is a slot into which a chemical array and/or chemical array slide holder is fitted. The chemical array holding element, or slot, may have any dimension but typically has a dimension that is adapted for receiving a chemical array and/or chemical array slide holder. For instance, in certain embodiments, a representative chemical array holding element may have a depth ranging from about 1 cm to about 16 cm, such as from about 2.5 cm to about 13 cm, including from about 5 cm to about 11 cm, e.g., about 7.5 cm. In one embodiment, a representative chemical array holding element may have a height ranging from about 1 cm to about 10 cm, such as from about 2.5 cm to about 13 cm,e.g., about 5 cm. In one embodiment, a representative chemical array holding element may have a width ranging from about .1 mm to about 5 cm, such as from about 1 mm to about 2.5 cm, or about 1 cm.

A chemical array container element may be configured to hold one or a plurality of chemical arrays and/or chemical array slide holders and therefore will be configured to contain one or more chemical array holding elements. In certain embodiments, the chemical array container element is configured for holding at least about 20 or more, such as at least about 48 or more, including at least 60 or more, e.g., at least 80 or more chemical array slides and/or slide holders and will therefore be configured to contain at least an equal amount of chemical array holding elements. By “chemical array slide” is meant a solid support, such as a substrate, that may comprise one or more arrays. Typically, a chemical array slide, used in conjunction with the subject invention, is made of silica, e.g., glass. Chemical array slides may have a width ranging from about 1 mm to about 200 mm, such as from about 10 mm to about 100 mm, including from about 25 mm to about 50 mm and a length ranging from about 15 mm to about 150 mm, such as from about 50 mm to about 100 mm, e.g., about 75 mm, and are typically adapted for being fitted within a chemical array slide holder.

By “chemical array slide holder” is meant an element that is configured for holding at least one chemical array slide and is adapted for being fitted within a chemical array holding element. For instance, a chemical array slide holder may be a micro array slide holder that, in representative embodiments, ranges in length from about 1 cm to about 16 cm, such as from about 2.5 cm to about 13 cm, including from about 5 cm to about 11 cm, e.g., about 7.5 cm. In certain embodiments, a chemical array slide holder may have a length ranging from about 1 cm to about 16 cm, such as from about 2.5 cm to about 7.5 cm, including from about 5 cm wide, and may have a thickness ranging from about .1 mm to about 5 cm, such as from about 1 mm to about 2.5 cm, e.g., about 1 cm. In one representative embodiment, a chemical array slide holder of the invention has a length of about 7.5 cm, a width of about 5 cm, and a thickness of about 1 cm, such as is available from Agilent (Palo Alto, Calif.). A chemical array slide holder may additionally include a clasping element for securing a chemical array slide to be contained.

Accordingly, in one embodiment, one or more chemical arrays are located on one or more chemical array slides that are held in place within the chemical array container element by one or more chemical array holding elements. Thus, in representative embodiments, each of: the chemical array container element, holding element, chemical array slide holder, and chemical array slide; will have compatible dimensions to one another so as to allow each element to effectively interact with one another and be entirely contained within the subject chemical array housing of the invention.

One feature of the chemical array container element is that it is configured to partially enclose a space (i.e., volume). By “partially enclose” is meant that the container element is configured for defining an interior environment of the housing from an external environment, wherein the interior environment may be substantially separated from the outside environment. For instance, in certain embodiments at least about 70%, at least about 60%, at least about 50%, at least about 40%, or at least about 30% of the container element is enclosed, e.g., by a bottom and side(s) of the container element. In one embodiment, the chemical array container element is open to an outside environment, but includes a plurality of chemical array holding elements that, when filled with chemical arrays or chemical array slide holders, will be substantially bounded on its side and bottom portions.

As reviewed above, in representative embodiments the container element of the housing is configured to receive (i.e., mate with) a lid element, e.g., so as to provide for a substantially completely enclosed space inside the chemical array housing structure. It is noted that although “top,” “bottom,” and “side” portions are indicated herein these are for ease of reference only as actual top, bottom, and side portions will depend on the configuration, placement, and use of the actual chemical array housing. For instance, in certain embodiments, the lid portion is configured for fitting onto a chemical array container element and thereby enclosing at least about 50% or more, at least about 60% or more, at least about 70% or more, at least about 80% or more, or at least about 90% or more, including all of the interior space of the housing.

In one embodiment, the lid element includes a slot element for facilitating the transfer of a chemical array from the interior of the chemical array housing to the chemical array reader, as will be described in greater detail below. In representative embodiments, such a slot element may range in length from about 1 cm to about 16 cm, such as from about 2.5 cm to about 13 cm, including from about 5 cm to about 10 cm, e.g., about 7.5 cm, and may range in width from about. 1 mm to about 5 cm, such as from about 1 mm to about 2.5 cm, e.g., about 1 cm.

In a representative embodiment, the chemical array housing lid element may be configured to fit onto the container element in such a manner as to create a substantially air-tight or sealed volume in the housing, where the volume in the housing is bounded by the interior wall(s) of the container element and the interior wall of the lid element. By substantial air tight seal is meant a seal that is effective for preventing the evacuation of at least about 60%, at least about 70%, at least about 80%, at least about 90%, or at least about 99% of one or more gasses from the inside environment of the housing through the interface where the lid contacts the chemical array container element. Such an air-tight interaction may be facilitated by one or more gaskets and/or sealants, as is well known in the art.

The lid element may or may not be readily separable from the container element. As such, in certain embodiments, the lid is readily removable from the container element, e.g., to provide access for initially loading of the housing with slides, etc. In such embodiments, any convenient type of securing means that can be open and closed may be employed, e.g., snap-fit, compression, screw, etc., securing elements may be employed. In yet other embodiments, the lid may not be readily removable or separable from the container, where the lid may be bonded to the secure, e.g., welded to the container, held in place relative to the container with an adhesive, etc

In one embodiment, the chemical array housing, including lid and container elements thereof, is adapted for being fit into a chemical array reader and is configured for rotating, so as to facilitate the delivery of the one or more chemical arrays to the chemical array reader for scanning. In such embodiments, the a lid element may be configured for moving, e.g., rotating, along with the chemical array container element, where in other embodiments, the lid may be configured for moving independently of the chemical array container element or at least not substantially moving as the container element moves, e.g., rotates. In another embodiment, the chemical array housing is configured for being removable from the array reader.

As reviewed above, another feature of the chemical array housing is the presence of a gas delivery element. A gas delivery element of the subject invention may be any shape and size, so long as it is configured for receiving a gas from an external source, e.g., a reservoir, and allowing the introduction of the gas to an environment defined by the inside of the housing, e.g., an area defined by the chemical array container and lid elements. In representative embodiments, the gas delivery element is configured to provide for delivery of a gas to at least a portion of, if not substantially all of or all of, the interior of the housing. A variety of different types of gas delivery elements may be present, where representative gas delivery elements include, but are not limited to: an opening or hole, a tubing, a fitting, a molded fitting, a valve, or the like, or a combination thereof. In one embodiment, the gas delivery element is simply an opening through which gas from an external gas source passes into the interior of the housing so as to deliver a gas to the environment within the housing. In yet another embodiment, gas delivery element is a tubing which passes from an environment outside of the chemical array housing into the interior of the chemical array housing. In certain of these embodiments, the tubing may wrap around at least a portion of the housing and be removably attached to a container element or a lid element of the housing. For instance, in one embodiment, tubing from an outside gas source enters the internal environment of the housing through an opening, wraps around at least a portion of the interior surface of a lid element of the housing, and is removably attached to the lid element of the housing, e.g., with a clasp or other convenient securing element. Additionally, one or more sections of the tubing may be perforated. For instance, a section of tubing that enters inside the housing may be perforated so as to facilitate the delivery of a gas to the interior environment of the housing. In certain embodiments in which the tube is perforated, the perforation pattern is spiral so that the gas flow out of the tubing is evenly directed over the arrays. With the spiral perforation there is no need to aim the gas flow from the tubing. In another embodiment, the gas delivery element is a molded fitting, which may contain a valve element, to which tubing from an external gas source connects. Additionally, a gas delivery element may be operably connected to a gas diffuser, tubing, and/or valve elements that are configured for facilitating the delivery of one or more gasses, such as nitrogen, dry air, or the like, from one or more external sources to the chemical array housing, as will be described in more detail below.

In certain embodiments, a chemical array housing of the invention may also include additional fittings, valves, dividers, chamber-elements, sensors, and the like. For instance, a chemical array housing may include one or more sensors for detecting and/or measuring an amount of one or more of a gasses (such as ozone), a chemical, an analyte, or the like within the interior of the chemical array housing. Suitable sensors are well known in the art and include, but are not limited to nitrogen sensors, ozone sensors, halide sensors, and the like.

As reviewed above, the gas delivery element may is associated with the housing, and may be associated with either the lid or container elements thereof. In representative embodiments, gas delivery element is associated with the lid element.

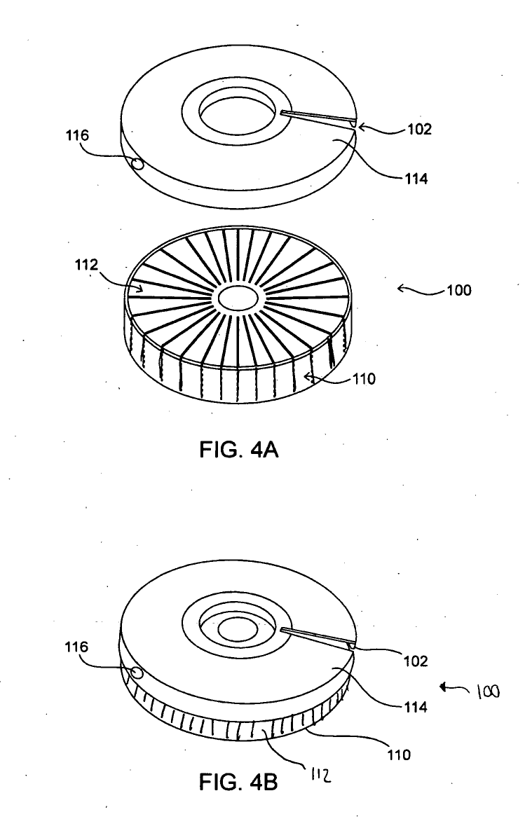

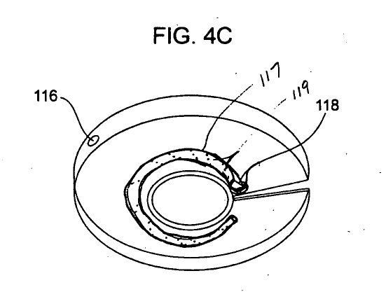

As mentioned above, in one representative embodiment, the chemical array housing is configured as a carousel. By “carousel” is meant a circular chemical array housing that is configured for holding one or more chemical arrays, such as is available from Agilent Technologies (Palo Alto, Calif.). Accordingly, a carousel for use with the subject invention contains a plurality of chemical array holding elements that are each configured for housing a substrate, such as a chemical array slide. For instance, with reference to FIG. 4A, a representative embodiment of a chemical array housing of the subject invention configured as a carousel is provided. It is to be noted that although the housing is depicted as being a circular carousel it is understood that the housing of the subject invention may be in any shape or size, as is known in the art, for instance, in the shape of a rectangular box wherein a plurality of chemical arrays are stacked one on top of the other.

Accordingly, when configured as a circular carousel, for example as depicted in FIG. 4A, the diameter of the carousel may vary depending on, for instance, the dimensions and the number of the chemical arrays to be housed. For instance, an embodiment of a chemical array carousel may have a diameter ranging from about 25 cm to about 45 cm, such as from about 30 cm to about 40 cm, including from about 30 cm to about 35 cm. A carousel configured in accordance with the subject invention may further have a height that is proportional to the width of the chemical arrays and/or chemical array holders to be housed by the carousel. For instance, a chemical array carousel may have a height ranging from about 1 cm to about 15 cm, such as from about 2.5 cm to about 7.5 cm, e.g. about 5 cm.

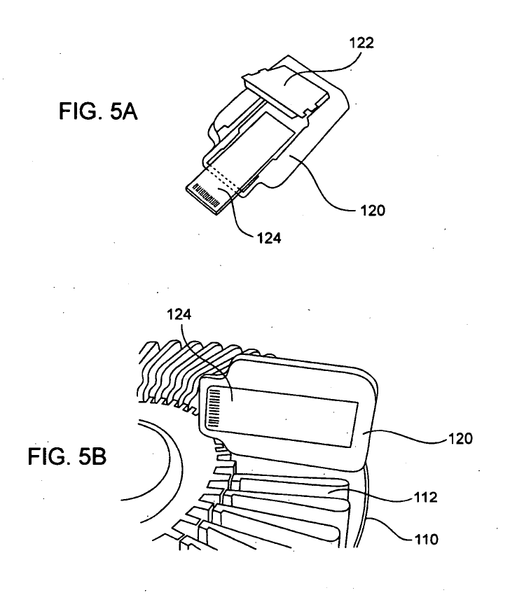

In accordance with the subject invention, the carousel 100 includes a container element 110 that is configured to contain a plurality of chemical array holding elements 112 that are adapted for housing one or more chemical arrays and/or chemical array slide holders (See FIGS. 5A and 5B). The chemical array carousel 100 includes a cover element 114, wherein, the cover element 114 contains a gas delivery element 116, that is configured as an opening through which a gas from an external source may be delivered (e.g., through tubing) to an inside environment of the carousel housing and contains a slot element 102, that is configured for facilitating the transfer of a chemical array from the carousel to the chemical array reader.

As can be seen in FIG. 4B, the cover element 114 is configured for fitting onto the container element 110 and thereby partially enclosing the container element 110.

FIG. 4C depicts the underside of the cover element 114. As can be seen in FIG. 4C, the cover element 114 includes an additional fitting element 118 that is adapted for receiving and connecting tubing to the cover element 114. In this embodiment, tubing from an outside gas source may enter the underside of the cover element 114 by a gas delivery element 116 and be connected to fitting element 118 so as to be secured to the lid element. As described above, once inside the cover element, delivery tubing 117 may wrap around the cover element 114 and be perforated (multiple holes 119 arranged in a spiral pattern) so as to facilitate the delivery of a gas from the external gas source. Accordingly, the cover element may include one or more securing elements, such as hooks, clamps, clasps, or the like for securing tubing to the underside of the cover element.

With reference to FIGS. 5A and 5B, FIG. 5A depicts a representative chemical array slide holder 120 for use in connection with the chemical array housing of the invention. Slide holder 120 is configured for receiving a chemical array substrate, such as a slide 124, and for securing the slide 124 within the slide holder 120 by securing element 122. As seen in FIG. 5B, slide holder 120, containing slide 124, is adapted for being fit into chemical array holding element 112, which is a part of chemical array container 110.

As reviewed above, a feature of the chemical array housing of the subject invention is that it is adapted for being fit into a chemical array reader and configured for receiving a gas from an external source. One or more gasses may be introduced into the chemical array housing for a multiplicity of purposes. For instance, a gaseous reagent or reagents may be introduced for the purpose of contacting the surface of the array, or one or more gasses may be introduced for the purpose of purging a gas, e.g., ozone from the chemical array housing, e.g., before or during reading. For example, in one embodiment a chemical array housing of the subject invention is configured for being purged by a gas. By purging is meant causing the evacuation of one or more gases present in the interior of the housing from the interior of the housing.

For instance, in one embodiment one or more relatively more reactive gases, such as but not limited to, ozone, halides, chlorine, other oxidants, and the like, is purged or evacuated by the addition of one or more less or non-reactive gases, such as, but not limited to, nitrogen, dry air, or the like. In this manner, the chemical array housing (or at least a portion thereof depending on when the determination of purging is made) may be substantially purged of reactive gasses, such as ozone, halides, and the like. By “substantially purged” is meant the evacuation of at least about 50%, at least about 60%, at least about 70%, at least about 80%, at least about 90%, or at least about 99% of gas, such as ozone, from the inside environment of the housing. In certain embodiments, substantially purging according to the subject protocols results in a reduction of a reactive gas, e.g., ozone, to a level that is sufficiently low as to not cause the negative action of the gas. For example, where ozone is the reactive gas, substantially purging according to the invention results in a reduction in the amount of ozone present in the chamber to a level such that it does not have a negative effect on the dyes present on arrays in the chamber. In certain embodiments, substantially purging according to the invention results in a reduction of the reactive gas to a concentration that is in the low parts per billion, e.g., less than about 10 ppb, such as less than about 5 ppb, including less than about 3 ppb, e.g., less than 1 ppb.

In certain embodiments, the chemical array housing may include an outlet valve and/or a vacuum connection so as to facilitate the purging of the one or more reactive gases. In certain embodiments, a gas may be introduced into the chemical array housing either before it is placed within a chemical array reader or introduced once the housing is placed within the reader but before use.

Where desired, e.g., where the lid and container elements make a substantially fluid and vapor tight sealed interior in the housing, in addition to the gas delivery element, one or more gas removal elements may be present, which element(s) provides for removal of gas from the interior of the housing. As with the gas delivery element, the gas removal element, when present, may vary considerably, where representative gas removal elements include openings, vents, tubing, etc.

In another aspect, the subject invention is directed to a chemical array reader that includes a chemical array housing, as described above. Any optical chemical array reader or device may be used so long as it is adapted to receive, and can be operatively linked to, a chemical array housing of the subject invention. Representative optical readers of interest include those described in U.S. Pat. Nos. 5,585,639; 5,760,951; 5,763,870; 6,084,991; 6,222,664; 6,284,465; 6,329,196; 6,371,370 and 6,406,849 the disclosures of which are herein incorporated by reference. In one embodiment, a suitable optical reader includes a chemical array housing of the subject invention and may include a gas conveyer element, for instance tubing, for conveying gas form an external source to said gas delivery element of the chemical array housing. The gas conveyer element may also include connectors for connecting the tubing to the external source and/or the chemical array housing. Additionally, the chemical array reader may include a back panel with fittings, such as bulk-head fittings, configured for conveying tubing from the external source to the chemical array housing. The external gas source may include a valve, such as a solenoid valve, that is configured for regulating or controlling the amount of gas to be delivered to the carousel. In one embodiment, the valve is controlled by a computer program, as described in more detail below.

The gas conveyer element may be any element configured for conveying one or more gasses from an external source to the chemical array housing of the subject invention, when present in the reader. In one embodiment, the gas conveyer element is tubing that is attached to an external gas source and/or attached to the chemical array housing by an airtight fitting that may include a valve, such as a solenoid valve, that is configured for opening and closing and thereby regulating or controlling the amount of gas that is delivered to the chemical array housing. The tubing may connect directly to the casing of the chemical array housing (i.e., to a side, bottom or a top portion), to a lid of the chemical array housing, or to the gas delivery element of the chemical array housing.

When the housing is present in the reader, e.g., during operation of the reader, a variety of gasses may be introduced into the chemical array housing, as desired. For instance, gasses such as, but not limited to, nitrogen, compressed air passed through a activated charcoal filter to remove any ozone, and the like may be introduced into the chemical array housing.

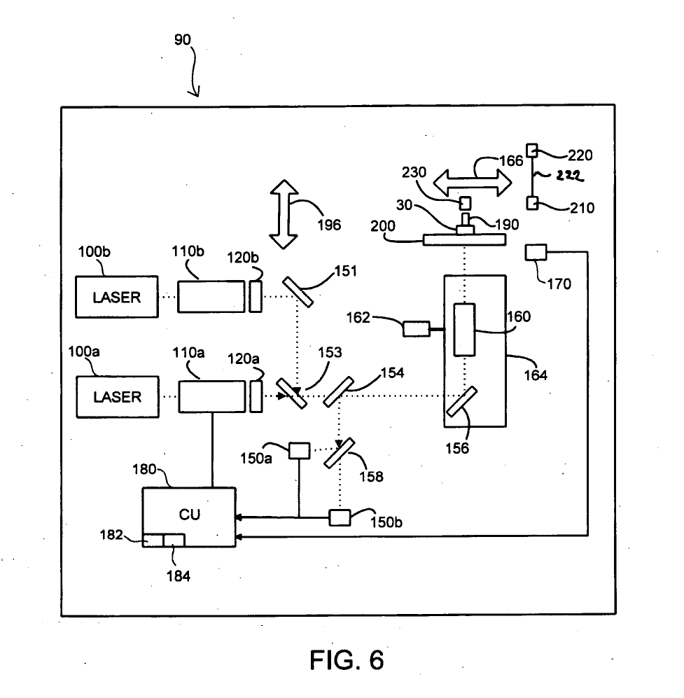

Referring now to FIG. 6, an apparatus 90 of the present invention (which may be generally referenced as an array “reader” or “scanner”) is illustrated. A multi-substrate chemical array housing 210 configured in accordance with the subject invention, as described in detail above, is loaded with one or more substrates 30, such as chemical array slides, and placed in the reader 90. The multi-substrate chemical array housing 210 may have one or more gasses introduced therein (e.g., be purged) prior to placement in the reader 90 or may have one or more gasses introduced therein once placed with in the reader. In the latter case, the chemical array housing 210 will be operatively linked to at least one gas conveyer element 222 that is configured for conveying one or more gasses to the chemical array housing 210 from at least one external source 220. The reader 90 contains automatic means 230 for removing a substrate 30 from the gas purged chemical array housing 210 and placing the substrate 30 onto the holder 200. This allows for the automatic scanning of one or more substrates that have previously been loaded into the chemical array housing 210. Once scanned, the automatic means 230 then removes the substrate 30 from the holder 200, returns the substrate 30 to the chemical array housing 210, and removes another substrate from the housing to be placed onto the holder for automatic scanning. The substrate 30 may be delivered to the holder 200 in the form of an array package and therefore may comprise one or more chemical arrays.

Once a substrate 30 is placed onto the holder 200, a scan system causes an illuminating region in the form of a light spot from each laser 100a, 100b, and a detecting region of each detector 150a, 150b (which detecting region will form a pixel in the detected image), to be scanned across multiple regions of an array package 30 mounted-on holder 200. The scanned regions for an array 112 will include at least the multiple features 116 of the array. In particular the scanning system is typically a line by line scanner, scanning the interrogating light in a line across an array 112 when at the reading position, in a direction of arrow 166, then moving (“transitioning”) the interrogating light in a direction into/out of the paper as viewed in FIG. 2 to a position at an end of a next line, and repeating the line scanning and transitioning until the entire array 112 has been scanned. This can be accomplished by providing a housing 164 containing mirror 156 and focuser 160, which housing 164 can be moved along a line of pixels (that is, from left to right or the reverse as viewed in FIG. 2) by a transporter 162. The second direction 196 of scanning (line transitioning) can be provided by second transporter which may include a motor and belt (not shown) to move holder 200 along one or more tracks. The second transporter may use a same or different actuator components to accomplish coarse (a larger number of lines) movement and finer movement (a smaller number of lines). The reader of FIG. 6 may further include a reader (not shown) which reads an identifier from an array package 30. When an identifier is in the form of a bar code, that reader may be a suitable bar code reader.

A light system provides light from a laser 100a or 100b which passes through an electro-optic modulator (EOM) 110a or 110b with attached polarizer 120a or 120b. Each laser 100a, 100b may be of different wavelength (for example, one providing red light and the other green) and each has its own corresponding EOM 110a, 110b and polarizer 120a, 120b. The beams may be combined along a path toward a holder 200 by the use of full mirror 151 and dichroic mirror 153. A control signal in the form of a variable voltage applied to each corresponding EOM 110a, 110b by the controller (CU) 180, changes the polarization of the exiting light which is thus more or less attenuated by the corresponding polarizer 120a, 120b. Controller 180 may be or include a suitably programmed processor. Thus, each EOM 110 and corresponding polarizer 120 together acts as a variable optical attenuator which can alter the power of an interrogating light spot exiting from the attenuator in a manner, and for purposes, such as described in U.S. Pat. No. 6,406,849, incorporated herein in its entirety by reference.

The remainder of the light from both lasers 100a, 100b is transmitted through a dichroic beam splitter 154, reflected off fully reflecting mirror 156 and focused onto either an array 112 of a substrate or array package 30 mounted on holder 200, or a calibration member (not shown), whichever is at a reading position, using optical components in beam focuser 160. Light emitted, in particular fluorescence, at two different wavelengths (for example, green and red light) from features 16, in response to the interrogating light, is imaged using the same optics in focuser/scanner 160, and is reflected off mirrors 156 and 154. The two different wavelengths are separated by a further dichroic mirror 158 and are passed to respective detectors 150a and 150b. More optical components (not shown) may be used between the dichroic and each detector 1 50a, 1 50b (such as lenses, pinholes, filters, fibers etc.) and each detector 150a, 150b may be of various different types (e.g. a photo-multiplier tube (PMT) or a CCD or an avalanche photodiode (APD)). All of the optical components through which light emitted from an array 112 or calibration member in response to the illuminating laser light, passes to detectors 150a, 150b, together with those detectors, form a detection system. This detection system has a fixed focal plane.

An autofocus detector 170 is also provided to sense any offset between different regions of array 112 when in the reading position, and a determined position of the focal plane of the detection system. An autofocus system includes detector 170, processor 180, and a motorized adjuster to move holder in the direction of arrow 196. A suitable chemical array autofocus system is described in pending U.S. patent application Ser. No. 09/415,184 for “Apparatus And Method For Autofocus” by Dorsel et al., filed Oct. 7, 1999, incorporated herein by reference, as well as European publication EP 1091229 published Apr. 11, 2001 under the same title and inventors.

Controller 180 of the apparatus 90 is connected to receive signals from detectors 150a, 150b (these different signals being different “channels”), namely a signal which results at each of the multiple detected wavelengths from emitted light for each scanned region of array 112 when at the reading position mounted in holder 200. Controller 180 also receives the signal from autofocus offset detector 170, and provides the control signal to EOM 110a and 110b, and controls the scan system. Parameter settings are entered and stored in Controller 180, and used by Controller 180 to perform a scan run. Controller 180 may also analyze, store, and/or output data relating to emitted signals received from detectors 150a, 150b in a known manner. Controller 180 may include a computer in the form of a programmable digital processor, and include a media reader 182 which can read a portable removable media (such as a magnetic or optical disk), and a communication module 184 which can communicate over a communication channel (such as a network, for example the internet or a telephone network) with a remote site (such as a database at which information relating to array package 30 may be stored in association with the identification 40). Controller 180 is suitably programmed to execute all of the steps required by it during operation of the apparatus, as discussed further below. Alternatively, controller 180 may be any hardware or hardware/software combination which can execute those steps.

In one aspect, the present invention is directed to a computer program that may be utilized to carry out the above steps. Therefore, the introduction of a gas into a chemical array housing, the loading of a chemical array from the chemical array housing into a chemical array reader, and the reading thereof by the reader in accordance with the present invention may be carried out under computer control, that is, with the aid of a computer. The computer may be driven by software specific to the methods described herein. Examples of software or computer programs used in assisting in conducting the present methods may be written in any convent language, e.g. Visual BASIC, FORTRAN and C++ (PASCAL, PERL or assembly language). It should be understood that the above computer information and the software used herein are by way of example and not limitation.

Programming according to the present invention, i.e., programming that allows one to introduce one or more gasses into a chemical array housing (e.g., a carousel) and preprogram a scanner based on the information obtained about specific substrates loaded into the chemical array housing can be recorded on computer readable media, e.g., any medium that can be read and accessed directly by a computer. Such media include, but are not limited to: magnetic storage media such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture that includes a recording of the present programming/algorithms for carrying out the above described methodology.

In certain embodiments, a processor of the subject invention may be in operable linkage, i.e., part of or networked to, the aforementioned device, and capable of directing its activities. A processor may be pre-programmed, e.g., provided to a user already programmed for performing certain functions, or may be programmed by a user. Thus, in certain embodiments, the programming is further characterized in that it provides a user interface, where the user interface presents to a user the option of selecting among one or more different, including multiple different, rules for individually controlling the introduction of at least one gas into the chemical array housing and/or pre-selecting scanning parameters. A processor may be remotely programmed by “communicating” programming information to the processor, i.e., transmitting the data representing that information as electrical signals over a suitable communication channel (for example, a private or public network). Any convenient telecommunications means may be employed for transmitting the programming, e.g., facsimile, modem, Internet, LAN, WAN or other network means, wireless communication, etc. In one aspect, the subject invention is directed to a method of reading or interrogating a chemical array on a substrate, such as a chemical array slide, that is delivered from a chemical array housing (e.g., a carousel) of the invention to a chemical array reader. In a representative mode of operation, an array to be scanned, e.g., that has been contacted with a sample of interest, is first inserted into an appropriate container element of a chemical array housing, the chemical array housing is fitted to a chemical array reader for reading. As summarized above, a gas may be introduced into the housing at any convenient time before, during and/or after the reading. Thus, in one embodiment, one or more gasses are introduced into the chemical array housing of the subject invention in a manner effective for facilitating the substantial purging of one or more gasses initially present in the housing, as described above. As such, one or more gasses may be introduced into a chemical array housing for the purpose of purging the chemical array housing of one or more reactive gasses such as ozone, or one or more gasses may be introduced for the purpose of contacting one or more chemical arrays with the gas. In certain of these embodiments, the gas, such as nitrogen, may be introduced into the interior of the housing in a manner sufficient to maintain the level of ozone in the housing to value of less than about 5 ppb, such as less than about 3 ppb including less than about 1 ppb for at least a period of time sufficient to read the arrays in the housing, e.g., for at least about 1 hrs, such as at least about 2 hrs, including at least about 4 hrs or longer.

Gas introduction may be a one time event, or may be a continual process that occurs throughout the entire scanning routine. Additionally, gas introduction may be controlled by a shut off valve, or the like, located on the external gas source, or attached to the chemical array housing, that may be turned on or off manually; may be regulated by an end user, via a computer program accessed through a Graphic User Interface (GUI); or may be regulated directly by a computer program in accordance with preset parameters, as will be described in greater detail below.

In representative embodiments, the methods described above are particularly useful in automatically scanning a plurality of substrates (e.g., biopolymeric array substrates or slides) where a user can load a chemical array housing or carousel with several substrates, purge the chemical array housing to reduce a level of a reactive gas, such as ozone, set the scanning parameters for each individual substrate, initiate the scan of one or more of the substrates, and leave the scanner unattended until all the selected substrates have been scanned.

Results from reading an array may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing). Stated otherwise, in certain variations, the subject methods may include a step of transmitting data from at least one of the detecting and deriving steps, to a remote location. The data may be transmitted to the remote location for further evaluation and/or use. Any convenient telecommunications means may be employed for transmitting the data, e.g., facsimile, modem, internet, etc.

In general the above methods are useful in reading a plurality of arrays, where the plurality of arrays have been loaded into a reader using a chemical array housing into which one or more gasses have been introduced, such as a rack or carousel. Chemical array housings that can be loaded with 2 or more, 4 or more, 8 or more, 15 or more, 24 or more, 48 or more, 64 or more, or 100 or more substrates are of particular suitability for use with the above methods, such as the one described above.

The subject chemical array housings and scanners, e.g., programmed according to the subject invention and holding a housing of the invention, find use in a variety of applications, where such applications are generally analyte detection applications in which the presence of a particular analyte in a plurality of samples is detected at least qualitatively, if not quantitatively. Specific analyte detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. References describing methods of using arrays in various applications include U.S. Pat. Nos. 5,143,854; 5,324,644; 5,288,644; 5,324,633; 5,432,049; 5,470,710; 5,492,806; 5,503,980; 5,510,270; 5,525,464; 5,547,839; 5,580,732; 5,661,028; 5,800,992-the disclosures of which are herein incorporated by reference.

Where the arrays are arrays of polypeptide binding agents, e.g., protein arrays, specific applications of interest include analyte detection/proteomics applications, including those described in U.S. Pat. Nos. 4,591,570; 5,171,695; 5,436,170; 5,486,452; 5,532,128 and 6,197,599 as well as published PCT application Nos. WO 99/39210; WO 00/04832; WO 00/04389; WO 00/04390; WO 00/54046; WO 00/63701; WO 01/14425 and WO 01/40803-the disclosures of which are herein incorporated by reference.

In another aspect, also provided are kits for use in practicing methods of the invention. Representative kits of the invention may include a chemical array housing that is configured for receiving a gas from an external source and adapted to be fit into a chemical array reader. For instance, a kit of the invention may include a chemical array container element, as described above, a lid, or both, wherein the container element, lid, or both may include a gas delivery element. In one embodiment, the kit may include a lid containing a gas delivery element, for instance, for retrofitting a chemical array housing, such as a carousel, that is already in the presence of a customer. In another embodiment, the kit may include a chemical array container element that includes a gas delivery element. In a further embodiment, the kit may include a chemical array housing that includes both a lid and a container element.

The kits may also include one or more of the following components: a gas conveyer element, configured for conveying gas from an external source to the housing; tubing (e.g., perforated tubing), configured for delivering one or more gases from an external source to the gas conveyer element; one or more valve components, configured for regulating or controlling the amount of gas to be delivered; one or more sensors, for detecting the level of a gas within the carousel; fitting and sealing components, for facilitating the interactions of the various other components; one or more external gas sources, configured for delivering one or more gases to the carousel; and computer programming for regulating or controlling the amount of gas to be delivered to the carousel from the external source.

Additional components necessary for practicing the subject methods may also be included as part of the kit, such as, micro array slides, slide holders, analyte detection assay components, such as sample preparation reagents, buffers, labels, denaturation reagents, wash mediums, labeled target nucleic acid sample, negative and positive controls, and the like.

Such kits may further include at least a computer readable medium including programming as discussed above and instructions. The instructions may include installation or setup directions. The instructions may include directions for use of the invention, with options or combinations of options as described above, and directions for conducting an assay. In certain embodiments, the instructions include both types of information.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or world wide web. Some form of access security or identification protocol may be used to limit access to those entitled to use the subject invention. As with the instructions, the means for obtaining the instructions and/or programming is generally recorded on a suitable recording medium.

Providing the chemical array housing, components, software and instructions as a kit may serve a number of purposes. The combination may be packaged and purchased as a means of upgrading an existing scanner. Alternately, the combination may be provided in connection with a new scanner in which the software is preloaded on the same. In which case, the instructions will serve as a reference manual (or a part thereof) and the computer readable medium as a backup copy to the preloaded utility.

It is evident from the above discussion that the subject invention provides an important breakthrough in the ability to scan large numbers of arrays automatically. Specifically, the subject invention allows one to scan a large. number of arrays automatically in a high througphut device using a chemical array housing where it is easy and convenient to control the environment of the interior of the housing, e.g., to reduce the effects of reactive gases such as ozone. Accordingly, the subject invention represents a significant contribution to the art.

All publications and patents cited in this specification are herein incorporated by reference, in their entirety, as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A chemical array housing, comprising:
at least one chemical array holding element; and
a gas delivery element for introducing a gas into said housing;
wherein said chemical array housing is configured to be used with an array reader.

2. The chemical array housing according to claim 1, wherein said housing comprises multiple chemical array holding elements.

3. The chemical array housing according to claim 1, wherein said chemical array holding element is configured to receive an array present in an array holder.

4. The chemical array housing according to claim 1, wherein said chemical array housing comprises a container element and a lid element.

5. The chemical array housing according to claim 4, wherein said lid element comprises an opening configured to provide for transfer of a chemical array into and out of said housing.

6. The chemical array housing according to claim 1, wherein said housing has a carousel configuration.

7. The chemical array housing according to claim 1, wherein said gas delivery element comprises a gas diffuser.

8. The chemical array housing according to claim 7, wherein said gas diffuser extends along at least a portion of an interior surface of said housing.

9. The chemical array housing according to claim 8, wherein said housing comprises a lid element and said diffuser extends along a least a portion of an interior surface of said lid element.

10. The chemical array housing according to claim 1, wherein said housing further comprises a valve that controls gas flow into said gas delivery element.

11. The chemical array housing according to claim 1, wherein at least one chemical array is present in a holder of said housing.

12. The chemical array housing according to claim 1, wherein said chemical array housing comprises substantially no ozone.

13. The chemical array housing according to claim 12, wherein said housing comprises nitrogen gas.

14. The chemical array housing according to claim 1, wherein said housing is configured to house 48 slides.

15. The chemical array housing according to claim 14, wherein said slides are each about 25 mm wide and about 75 mm long.

16. The housing according to claim 1, wherein said housing is configured for being removably positioned inside of said reader.

17. A chemical array reader comprising a housing according to claim 1.

18. The chemical array reader according to claim 17, wherein said housing is present inside of said reader.

19. The chemical array reader according to claim 18, further comprising a gas conveyer element for conveying gas to said gas delivery element of said housing.

20. The chemical array reader according to claim 19, wherein said gas conveyer element comprises tubing.

21. The chemical array reader according to claim 20, wherein said tubing is perforated.

22. The chemical array reader according to claim 20, wherein said gas conveyer element further comprises connectors for connecting said tubing to an external gas source.

23. The chemical array reader according to claim 22, wherein said gas conveyer element further comprises a valve.

24. The chemical array reader of claim 17, further comprising programming for regulating the delivery of gas into said housing.

25. The chemical array reader of claim 17, further comprising a sensor for detecting the presence of a gas in said housing.

26. A method for reading a chemical array, said method comprising:
a) providing a chemical array reader that comprises a housing according to claim 1 that includes a chemical array; and b) reading said chemical array with said chemical array reader.

27. The method according to claim 26, wherein said method further comprises introducing an ozone free gas into said housing through said gas delivery element of said housing.

28. The method of claim 27, wherein said ozone free gas is nitrogen.

29. The method according to claim 27, wherein said ozone free gas is compressed air that has had ozone removed from it by activated charcoal filter.

30. A kit comprising a housing according to claim 1.

31. The kit according to claim 30, wherein said kit further comprises a gas conveyer element for conveying gas from an external source to said housing.

32. A chemical array housing lid element, wherein said lid element comprises:
a) a lid configured for at least partially enclosing a chemical array container element, wherein said container element includes at least one chemical array holding element; and
b) a gas delivery element.

33. The chemical array housing lid element of claim 32, wherein said container element comprises a chemical array carousel.

34. The chemical array housing lid element of claim 33, wherein said gas delivery element comprises an opening configured for receiving a gas conveyer element.

35. The chemical array housing lid element of claim 34, wherein said gas conveyer element comprises tubing.

36. The chemical array housing lid element of claim 32, wherein said lid element further comprises a slot element configured for facilitating the delivery of a chemical array from said chemical array holding element to a chemical array reader.
